(19) United States
(12) Patent Application Publication
(10) Pub. No.: US 2019/0223835 A1
(43) Pub. Date: Jul. 25, 2019

(54) ULTRASOUND CATHETER WITH ENLARGED APERTURE

(71) Applicant: Mirsaid Seyed-Bolorforosh, Thousand Oaks, CA (US)

(72) Inventor: Mirsaid Seyed-Bolorforosh, Thousand Oaks, CA (US)

(21) Appl. No.: 15/980,597

(22) Filed: May 15, 2018

Related U.S. Application Data
(60) Provisional application No. 62/620,400, filed on Jan. 22, 2018.

(51) Int. Cl. A61B 8/00; A61B 8/12; A61B 8/08

(52) U.S. Cl. CPC A61B 8/445; A61B 8/483; A61B 8/4483; A61B 8/12

(57) ABSTRACT

Described herein is an intracardiac ultrasound catheter (IUC) with an enlarged distal end comprising an enlarged ultrasound transducer array for improved imaging. In some embodiments, the distal end and transducer array are foldable. In some embodiments, the transducer array is two-dimensional. The transducer array can be side-firing or end-firing. In some embodiments, the transducer array can be covered in its folded position by a catheter housing. In some embodiments, the transducer array can be covered in its folded position by a retractable distal end cover.

### RELATED APPLICATION

This application claims priority to U.S. Provisional Application No. 62/620,400, filed on Jan. 22, 2018, which is incorporated by reference herein in its entirety.

### BACKGROUND OF THE INVENTION

### Field of the Invention

Embodiments of the present invention relate to catheters and, more specifically, to intracardiac ultrasound catheters (IUC) with enlarged apertures for improved imaging.

### Description of the Related Art

Intracardiac ultrasound catheters (IUCs) are used for visualization and guidance of interventional cardiac surgical procedures, for example percutaneous closure which is a surgical procedure used to treat patients with patent foramen ovale (PFO) and atrial septal defect (ASD) where IUCs are used for guidance and visualization of PFO and ASD procedures. This allows a safe and effective catheter-based closure.

Another example of a procedure in which an IUC is used is left atrial appendage closure (also known as LAA closure or LAAC) which is a minimally invasive procedure that is used to reduce the risk of stroke that comes as a result of atrial fibrillation (also known as Afib or AF). The LAA procedure can be conducted under the imaging and guidance of IUC devices.

The imaging resolution of ultrasound imaging systems is directly proportional to the product of the aperture size and the operation frequency of the ultrasound transducer. However, for intracardiac applications there is a limit for the size of the catheter due to size of vessels which the catheter has to be deployed and passed through. This results in limited resolution and limited penetration for the IUC which is a current technology limitation. The typical size of an IUC is 8 French (2.7 mm diameter) to 10 French (3.3 mm diameter). This puts a limit on the imaging resolution and penetration of IUC devices. As shown in FIG. 1, the prior art catheter 100 comprises a body 102 with transducer array elements 104 along the length of the IUC at a distal end of the body and a signal cable 106 electrically and communicably connected to the transducer array elements. The transducer array elements 104 comprise a horizontal length and a vertical width, wherein the length of the transducer array elements (also known as the azimuth 110) is substantially parallel to the length of the body 102 and the width of the transducer array elements (also known as the elevation 112) is substantially perpendicular to the length of the body. As shown in FIG. 1, the elevation of the transducer array elements 104 is confined in size by the diameter of the catheter body 102 and, thus, the possible size of the transducer array elements 104 is restricted.

### SUMMARY

Described herein are embodiments of an intracardiac ultrasound catheter (IUC) comprising an enlarged aperture for two-dimensional (2D) and three-dimensional (3D) intracardiac imaging. The catheter comprises a body and an enlarged distal end that allows for an enlarged ultrasound transducer array with an enlarged aperture. The enlarged aperture allows for improved imaging, for example improved contrast, detailed resolution, imaging penetration, and far field elevation resolution. The enlarged transducer array also allows space for additional array elements to be included on the distal end.

The embodiments described herein are a catheter, which comprises: an elongated body, a distal end coupled to the elongated body, and a plurality of ultrasound transducer elements on the distal end, wherein a diameter of the distal end is greater than a diameter of the elongated body.

In some embodiments, the distal end of the catheter has a larger cross section and diameter than the cross section and diameter of the body of the catheter. The size of the catheter is limited by the size of the vessels in which the catheter is deployed and must pass through. The body of the catheter is longer than the body of the distal end. The body of the catheter can be about 120 cm to 150 cm long, is smaller in diameter than the distal end, which can be about 2 cm to 4 cm long. In some embodiments, the distal end comprises an enlarged transducer array with an enlarged aperture.

In some embodiments, the distal end of the catheter and an enlarged transducer array that it comprises can be expanded and contracted such that the diameter of the distal end, and the transducer array it comprises, is smaller when contracted than when expanded. In the contracted position, the distal end can be comparable in diameter to the body of the catheter, which keeps the full expanded size of the distal end from obstructing the catheter's deployment and passage through vessels. Once the catheter is positioned at the desired destination, the distal end is expanded and the enlarged transducer array with its enlarged aperture is able to obtain improved imaging information.

In some embodiments, the expandable distal end and transducer array are confined within a catheter housing while in its contracted position. The catheter housing can comprise an open or openable end from which the distal end can extend out of to expand. Once the images have been obtained, the distal end can retract back into the catheter housing to return to its contracted position before the catheter is withdrawn from the patient.

In some embodiments, the catheter has a distal end cover retractably coupled to the catheter housing such that the cover can extend past the end of the length of the catheter housing and retract to be positioned over the catheter housing. During the catheter deployment and positioning, the expandable distal end and transducer array are confined to their contracted position by the distal end cover that is extended past the end of the catheter housing, which does not extend over the distal end. To be expanded, the distal end cover retracts from the distal end and is positioned over the catheter housing, allowing the distal end and transducer array to expand. For removal of the catheter, the distal end retracts into the catheter housing, which confines the distal end and transducer array to their contracted position.

### BRIEF DESCRIPTION OF THE DRAWINGS

FIG. 1 is a side view of a catheter known in the field.

FIG. 2 is a side view of a catheter according to an embodiment of the present disclosure.

FIGS. 3a and 3b are side views of a catheter according to an embodiment of the present disclosure.

FIGS. 4a-4c are side views of a catheter according to an embodiment of the present disclosure.

FIG. 5 is a side view of a catheter according to an embodiment of the present disclosure.

FIGS. 6a and 6b are perspective side views of a catheter according to an embodiment of the present disclosure.

FIG. 7 is a perspective side view of a catheter according to an embodiment of the present disclosure.

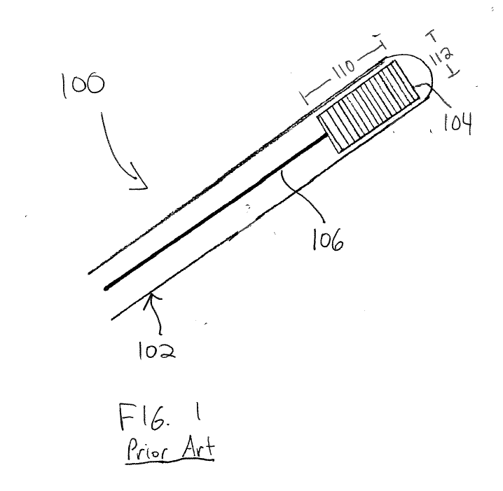

### DETAILED DESCRIPTION OF THE INVENTION

Embodiments incorporating features of the present invention include imaging catheters, for example intracardiac ultrasound catheters (IUCs), with enlarged apertures for two-dimensional (2D) and three-dimensional (3D) intracardiac imaging. The enlarged aperture allows for improved imaging, for example improved contrast, detailed resolution, imaging penetration, and far field elevation resolution.

For some embodiments, a distal end of the catheter can have a diameter of approximately 14 French to 16 French (a diameter of approximately 4.7 mm to 5.3 mm), while the length of the stiff portion of the distal end can remain close to current ICE catheters designs, i.e. approximately 2 cm to 4 cm. In some embodiments, the distal end can have a length of 9 cm or less. The transducer array, and thus the aperture, has a diameter approximately equal to the distal end's diameter. This results in about 40% to 60% larger elevation aperture size which will significantly improve image resolution and penetration.

The present invention is described herein with reference to certain embodiments, but it is understood that the invention can be embodied in many different forms and should not be construed as limited to the embodiments set forth herein. In particular, embodiments of the present invention are described below in regards to ultrasound catheters with enlarged apertures.

It is understood that when an element can be referred to as being “on” another element, it can be directly on the other element, or intervening elements may also be present. Furthermore, relative terms such as “inner”, “inside”, “inward”, “outer”, “outside”, “outward”, “upper”, “above”, “lower”, “beneath”, “below”, and similar terms, may be used herein to describe a relationship of one element to another. It is understood that these terms are intended to encompass different orientations of the device in addition to the orientation depicted in the figures.

It is understood that the term “azimuth” means the horizontal length of the ultrasound transducer array and/or elements. It is understood that the term “elevation” means the vertical height of the ultrasound transducer array and/or elements.

It is understood that the term “transducer array” is used to mean “ultrasound transducer array” unless specifically stated otherwise.

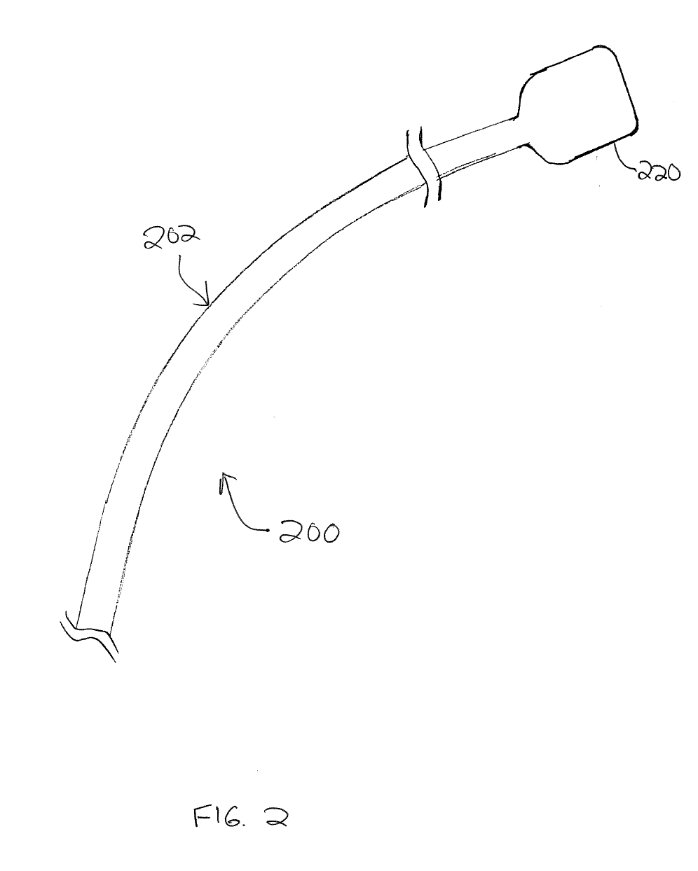

FIG. 2 shows a catheter 200 comprising a body 202 that is elongated with an enlarged distal end or tip 220 configured to comprise enlarged ultrasound transducer array elements (not shown). The catheter 200 comprises a signal cable 106 running along the length of the body 202 that is electrically and communicably connected to the transducer array elements (not shown). Although the catheter 200 shown in FIG. 2 cylindrical or near-cylindrical, the catheter 200 may be of any shape known within the field. In some embodiments, the enlarged distal end 220 is not at the end of the catheter 200, but rather a portion of the catheter extends from either side of the distal end.

The size of the body 202 and the distal end 220 are sized to successfully travel within the intended vessels. As the body 202 is longer than the distal end 220, the body can have a smaller size than the distal end 220 while maintaining the catheter's 200 overall mobility. In some embodiments, the size of the body 202 is approximately 8 to 10 French (a diameter of approximately 2.6 mm to 3.3 mm) and the distal end 220 is approximately 12 to 16 French (a diameter of approximately 4 mm to 5.3 mm). In some embodiments, the size of the body can be approximately 6 to 8 French (a diameter of approximately 1.3 mm to 2.7 mm). In other embodiments, the size of the body can be approximately 4 to 6 French (a diameter of approximately 4 mm to 2.0 mm). In some embodiments, the size of the distal end 220 can be approximately 10 to 12 French (a diameter of approximately 3.3 mm to 4.0 mm). In other embodiments, the size of the distal end 220 can be approximately 16 to 18 French (a diameter of approximately 5.3 mm to 6.0 mm).

The enlarged distal end 220 allows for the catheter 200 to comprise enlarged transducer array elements. For example, the enlarged distal end 220 provides space for transducer array elements with a greater elevation. In some embodiments, the enlarged distal end 220 provides space for additional transducer array elements in the elevation and azimuth directions for two-dimensional and quasi two-dimensional imaging. The enlarged transducer array elements allows for a larger aperture for improved imaging.

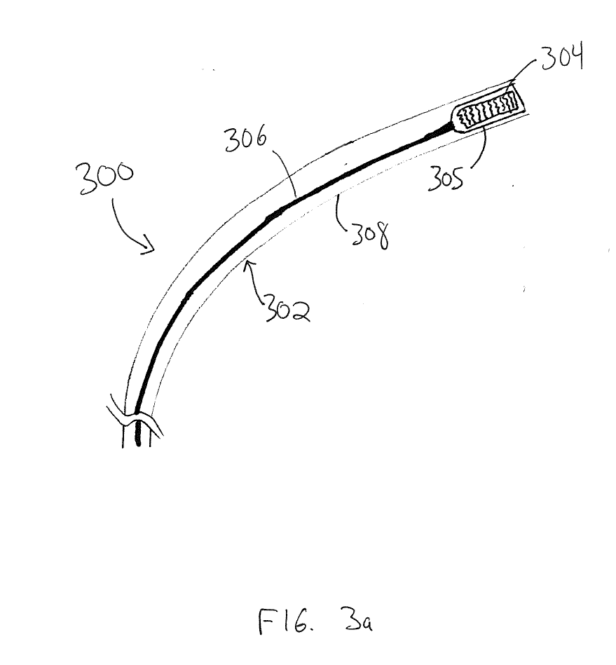

FIGS. 3a and 3b show a catheter 300 with an enlarged ultrasound transducer array 304 along the length of the catheter body 302 at the distal end 305. Although the embodiment shown in FIGS. 3a and 3b show a transducer array 304 that is side-firing, some embodiments can have a transducer array located at the end of the distal end 305 so that it is end-firing. The sizes of the body 302 and the distal end 305 can be comparable to the sizes of the body 202 and the distal end 220 of the catheter 200. The size of the transducer array 304 can be approximately the same size as the distal end 305. The size of the transducer array 304 allows for the catheter 300 to have a large aperture, which allows high resolution imaging as well as deep imaging penetration. Further, the catheter 300 allows for three-dimensional imaging since transducer array elements can reside in the elevation and azimuth direction of the aperture, forming a two-dimensional transducer array 304. In some embodiments, the catheter 300 comprises more than one transducer array 304, which can produce two or more imaging planes.

The transducer array 304 and distal end of the body 305 comprise one or more materials that can be folded or bent without damaging the structural integrity of the one or more materials. The distal end 305 and the transducer array 304 can have pre-formed folds to fold along when in the contracted position. In some embodiments, the distal end 305 and the transducer array do not have pre-formed folds but instead can fold at various positions. The transducer array 304 and distal end 305 can be biased toward an expanded position, as shown in FIG. 3b, such that the transducer array 304 and distal end 305 are in the expanded position unless a sufficient force is applied to them to contract them. In some embodiments, the transducer array 304 can be mounted on an umbrella device that is biased toward an expanded position. The umbrella device can be biased by mechanical components, including but not limited to springs or other biasing components known within the field, or by a memory material comprised in at least part of the umbrella device.

The transducer array 304 is electrically and communicably connected to a signal cable 306 that runs along the length of the body 302 and electrically and communicably connects the transducer array to a system connector (not shown). The signal cable 306 comprises interconnect wires and/or electronic beamforming components. The signal cable 306 can comprise one or more individual micro-coaxial cables or electrodes patterned on a soft substrate such as a thin layer of Kapton (Polyimide). The signal cable 306 can also comprise guide wires which run through the catheter 300 into the distal end 305 for controlling and manipulating the distal end once the catheter is at the imaging site.

The transducer array 304 can comprise micro machined ultrasonic transducers (CMUTs), piezoelectric micro machined ultrasonic transducers (PMUTs), or a different type of transducer. The transducer array can be made of one or more of the following piezoelectric materials: lead zirconate titanate (PZT), lead magnesium niobite-lead titanate (PMN-PT), and polyvinylidene difluoride (PVDF).

FIG. 3a shows the catheter housing 308 at least partially covering the body 302 including the distal end 305 and the transducer array 304, confining the distal end and the transducer array to a contracted position such that the distal end 305 and the transducer array 304 are not expanded to their full size. The catheter housing 308 can have an open end or an openable end by the distal end 305. The catheter housing 308 can be made of one or more biocompatible polymers, including but not limited to polyether block amide (PEBA), polyurethane, polyethylene, nylon, and polyester. In some embodiments, the distal end 305 is not at the end of the catheter 300, but rather a portion of the catheter extends from either side of the distal end.

While the catheter 300 is being moved through a patient's blood vessels, the distal end 305 and the transducer array 304 can remain confined within the housing 308 so that the full size of the distal end 305 and transducer array 304 is not an obstacle for positioning the catheter 300. Once the catheter 300 reaches its intended position, the distal end 305 and transducer array 304 are extended out of the end of the catheter housing 308 and expand. For example, for Intracardiac Echo, the IUC is typically placed in one of the four chambers of the heart. Once the catheter 300 is placed inside the chamber, then the distal end 305 and transducer array 304 are extended out of the catheter housing 308 with expanded aperture to allow high resolution and high penetration imaging. The transducer array 304 will be in the form of a conformal aperture, which allows the transducer array 304 to collapse and fit into the catheter housing 308 as well as expand for imaging to provide a large aperture.

FIG. 3b shows a catheter 300 with the catheter housing 308 retracted from the distal end 305, leaving the transducer array 304 and the distal end uncovered and in their expanded position. When uncovered, the transducer array can expand to its full size, which is substantially larger than the body 302 and comprises additional space in the azimuth 310 and the elevation 312 directions. The size of the transducer array 304 can be of any size that permits full expansion while located in the intended site. In some embodiments, the size of the transducer array 304 can have a width of approximately 1 cm and a length of approximately 1.5 cm. In some embodiments, the transducer array 304 can have a width of approximately 0.7 cm to 1 cm. In some embodiments, the transducer array 304 can have a width of approximately 1 cm to 1.7 cm. In some embodiments, the transducer array 304 can have a length of approximately 0.7 cm to 1 cm. In some embodiments, the transducer array 304 can have a length of approximately 1 cm to 1.7 cm.

A conformal and biocompatible material can be used as a substrate to hold the transducer elements in place, which allows the transducer array 304 to fold and unfold. For example, a thin polyimide sheet can be used as the substrate. Then the transducer elements that form the transducer array 304 can be placed on the substrate with electrical connections running through the polyimide substrate. The transducer elements are a two-dimensional array in order to allow for the aperture to be folded. The electrical connection to the transducer elements can be a one-dimensional array with elements electrically connected along the elevation aperture to form a one-dimensional array. The substrate can be a shaped-memory-polymer, which allows the material to change from its folded and deformed state while inside the catheter housing 308 to its original flat shape suited for imaging in its expanded position.

In a preferred embodiment, the piezoelectric material is Polyvinylidene DiFluoride (PVFD), which is a conformal piezoelectric material. The elements on the PVDF array are defined by forming the electrodes on the PVDF sheet. The PVDF sheet can be supported by a supporting substrate to control the shape of the array inside and outside the catheter housing 308. The elements on the transducer array 304 can be one-dimensional or two-dimensional arrays allowing two-dimensional or three-dimensional imaging, respectively.

Although PVDF is a good detector for ultrasound waves in fluid, it may not be an efficient transmitter. Therefore, in some embodiments, a second array (one-dimensional, 1.5-dimensional or two-dimensional) made of piezo-ceramic materials can be used for transmission while the reception of ultrasound can be formed using the PVDF or other conformal aperture designs.

As the distal end 305 is not supported by a rigid substrate in some embodiments, one challenge with the design of conformal arrays can be the accurate registration of the relative position of the transducer elements in the transducer array 304 required to properly beam-form during ultrasound transmission and reception. The structure under the array is designed in a manner to ensure accurate relative placement of elements on the array. In case there are errors in the relative position of elements on the array, this can be compensated by adaptively analyzing the relative phase of adjacent elements as a function of depth to form a coherent signal between adjacent elements from the same target to correct for any potential beam-forming error. An example of a way to measure potential phase errors is by comparing the backscatter radio frequency signal from adjacent elements and comparing the relative delay/phase between radio frequency signals to determine the required time delay correction. The beamforming electronics can be placed inside the catheter housing 308 or at the distal end 305 in a separate re-usable section of the catheter assembly.

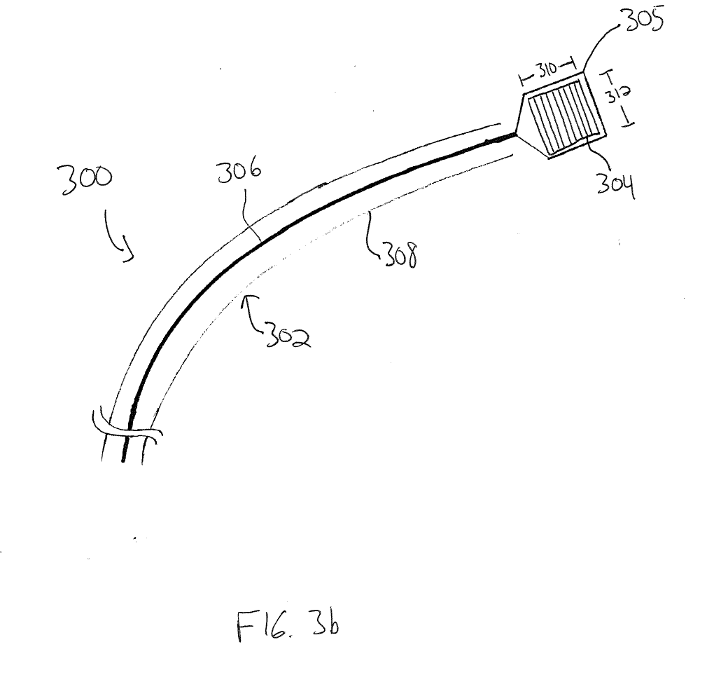

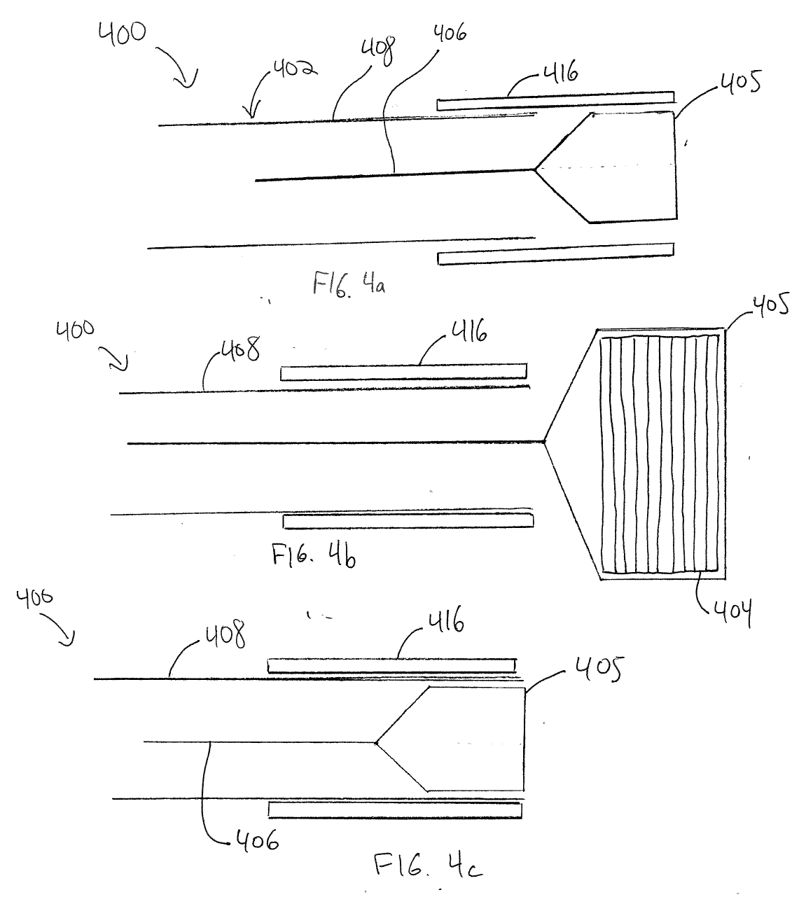

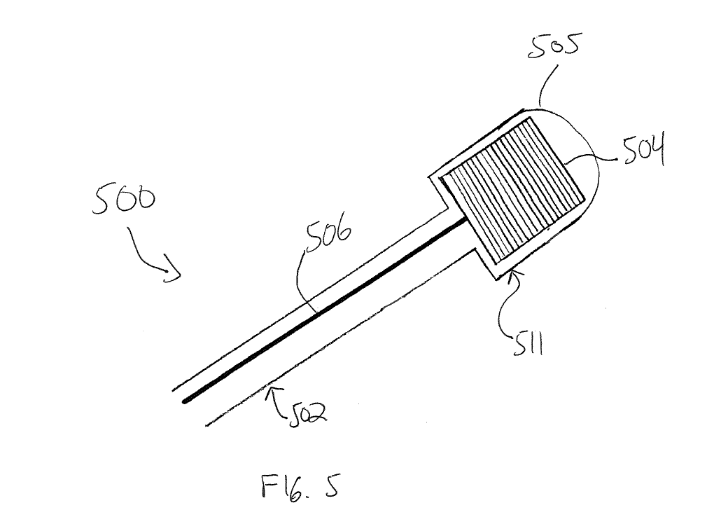

FIGS. 4a-4c show a catheter 400. Similar to the catheter 300, the catheter 400 comprises a body 402, an expandable/contractible transducer array 404 along the length of the distal end of the body 405, a signal cable 406, and a catheter housing 408. The size of the transducer array 404 and distal end 405 can correspond to the size of the transducer array 304 and distal end 305 of the catheter 300. In addition, the catheter comprises a distal end cover 416. FIG. 4a shows the catheter 400 in a state for guiding through a patient's vessels, wherein the transducer array 404 and the distal end 405 are held in the contracted position by the distal end cover 416. The distal end cover 416 at least partial covers the transducer array 404 and the distal end 405 and retractably couples to the catheter housing 408.

FIG. 4b shows the catheter 400 with an expanded transducer array 404. Once the catheter 400 is at the desired site, then the distal end cover 416 is retracted along the length of the body 402 in a direction away from the transducer array 404 so the transducer array 404 and the distal end 405 are no longer covered by the distal end cover 416, allowing the transducer array 404 and the distal end 405 to expand.

FIG. 4c shows the catheter 400 in its removal state. Once the imaging is completed, then the transducer array 404 can be pulled back into the catheter housing 408, wherein the catheter housing presses the transducer array 404 and distal end 405 to collapse them and fit them into the catheter housing which will in turn allow the removal of the entire catheter 400 from the patient.

FIG. 5 shows a catheter 500 with a body 502, a transducer array 504 on a distal end of the body 505, and a signal cable 506. The distal end 505 is enlarged and can have a size corresponding to the distal end 220 of the catheter 200 shown in FIG. 2, with the addition that an enlarged ultrasound transducer array 504 is on the distal end 505 along the length of the body 502. Although the embodiment shown in FIG. 5 shows a transducer array 504 that is side-firing, some embodiments can have a transducer array located at the end of the distal end 505 so that it is end-firing. The catheter 500 further comprises a catheter housing 511 that encases the body 502 and at least partially encases the distal end 505 and the transducer array 504. In some embodiments, the catheter housing 511 fully encases the distal end 505 and the transducer 504. In other embodiments, the distal end 505 and the transducer 504 are not encased by a catheter housing 511.

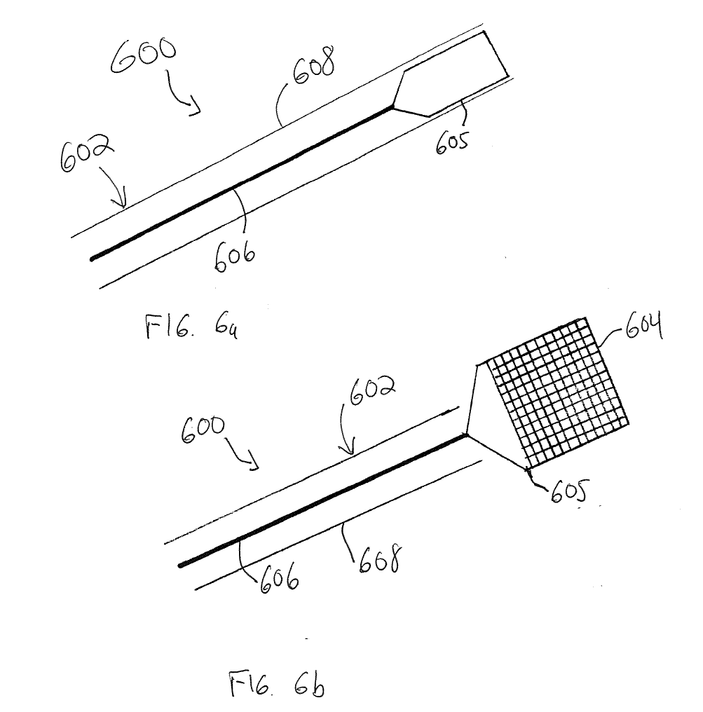

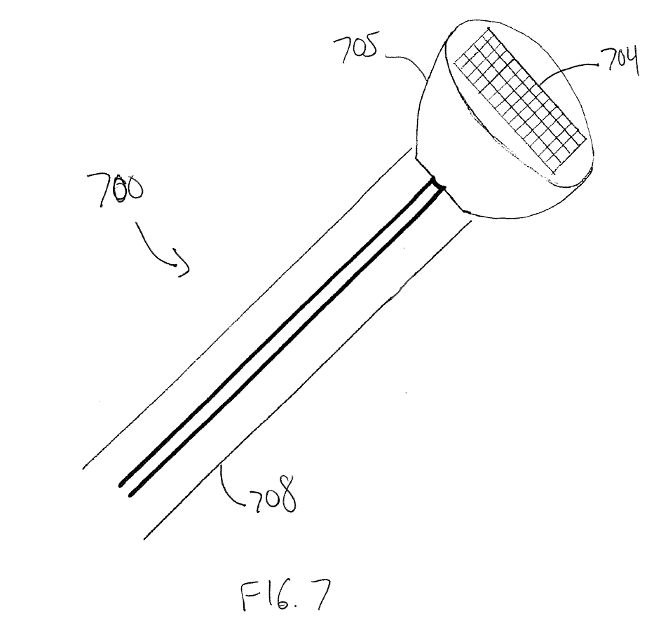

FIGS. 6a and 6b show a catheter 600 similar to the catheter 300 shown in FIGS. 3a and 3b, comprising a body 602, an transducer array 604, a distal end of the body 605, a signal cable 606, and a catheter housing 608 over at least part of the length of the body 602 in which the transducer array 604 and the distal end 605 can fold inside to be in a contracted position. The size of the transducer array 604 can correspond to the size of the transducer array 304 of the catheter 300. Unlike the catheter 300, the catheter 600 is end firing, having the transducer array 604 at the end of the distal end 605, such that the transducer array 604 is substantially perpendicular to the length of the body 602.

FIG. 6a shows the catheter 600 with the transducer array 604 and the distal end 605 in a contracted position within the catheter housing 608. FIG. 6b shows the catheter 600 with the transducer array 604 and the distal end 605 extended out of the housing 608 and in an expanded position. When the transducer array 604 is extended out of catheter housing 608, it expands like an umbrella forming a quadrilateral aperture. Similar to the catheter 400, in some embodiments the catheter 600 further comprises a distal end cover retractably coupled to the catheter housing 608.

FIG. 7 shows a catheter 700 similar to the catheter 600, but when the transducer array 704 and the distal end 705 are extended out of the catheter housing 708, they expand like an umbrella forming a circular aperture. The elements on the surface of transducer array 604 can be two-dimensional, one dimensional, or just two one-dimensional arrays arranged in an orthogonal manner. Once the imaging procedure is completed, the array is closed and retracted back into the catheter house to prepare for extraction out of the patient.

All the embodiments described herein can comprise a one-dimensional transducer array, a two-dimensional transducer array, or a transducer array with a design in between these such as a cross-shaped array. The transducer array can be rectangular including square or a parallelogram, circular, oval, triangular, pentagonal, hexagonal, heptagonal, octagonal, any polygonal, or any polygonal with one or more curved sides.

Although the present invention has been described in detail with reference to certain preferred configurations thereof, other versions are possible. Embodiments of the present invention can comprise any combination of compatible features shown in the various figures, and these embodiments should not be limited to those expressly illustrated and discussed. Therefore, the spirit and scope of the invention should not be limited to the versions described above.

We claim:

1. A catheter, comprising:
an elongated body;
a distal end coupled to said elongated body; and
a plurality of ultrasound transducer elements on said distal end;
wherein a diameter of said distal end is greater than a diameter of said elongated body.

2. The catheter according to claim 1, wherein said plurality of ultrasound transducer elements forms a transducer array, wherein a length of said transducer array is greater than said diameter of said elongated body.

3. The catheter according to claim 2, where a width of said transducer array is greater than said diameter of said elongated body.

4. The catheter according to claim 1, wherein said distal end is approximately 10 to 18 French.

5. The catheter according to claim 1, wherein said plurality of ultrasound transducer elements forms a two-dimensional transducer array.

6. The catheter according to claim 1, wherein said plurality of ultrasound transducer elements forms a plurality of transducer arrays.

7. The catheter according to claim 1, wherein said distal end is foldable.

8. The catheter according to claim 1, wherein a portion of said elongated body extends from either side of said plurality of ultrasound transducer elements.

9. An ultrasound catheter, comprising:
an elongated body;
a distal end coupled to said elongated body; and
a plurality of ultrasound transducer elements on said distal end;
wherein a diameter of said distal end is greater than a diameter of said elongated body, wherein said distal end is foldable.

10. The ultrasound catheter of claim 9, wherein said plurality of ultrasound transducer elements are side-firing.

11. The ultrasound catheter of claim 9, wherein said plurality of ultrasound transducer elements are end-firing.

12. The ultrasound catheter of claim 9, wherein said plurality of ultrasound transducer elements forms a two-dimensional transducer array.

13. The ultrasound catheter according to claim 9, wherein said plurality of ultrasound transducer elements forms a plurality of transducer arrays.

14. The ultrasound catheter of claim 9, further comprising a transduction material, wherein said transduction material comprises polyvinylidene difluoride.

15. The ultrasound catheter of claim 9, further comprising a transduction material, wherein said transduction material comprises a micro-machined capacitive sensor.

16. The ultrasound catheter of claim 9, further comprising a transduction material, wherein said transduction material comprises a micro-machined piezoelectric sensor.

17. The ultrasound catheter of claim 9, wherein said plurality of ultrasound transducer elements forms a foldable transducer array.

18. An ultrasound catheter, comprising:
an elongated body comprising a foldable portion, said foldable portion configured to expand and contract such that when said foldable portion is expanded a diameter of said foldable portion is greater than said elongated body, and when said foldable portion is contracted said diameter of said foldable portion is approximately equal to said diameter of said elongated body; and
a plurality of ultrasound transducer elements on said foldable portion.

19. The ultrasound catheter of claim 18, wherein said plurality of ultrasound transducer elements forms a two-dimensional transducer array.

20. The ultrasound catheter according to claim 18, wherein said plurality of ultrasound transducer elements forms a plurality of transducer arrays.
